(19) United States
(12) Patent Application Publication
(10) Pub. No.: US 2008/0167563 A1
(43) Pub. Date: Jul. 10, 2008

(54) Non-invasive blood flow monitor

(75) Inventor: Titi Trandafir, S. Plainfield, NJ (US)

Correspondence Address: CARTER, DELUCA, FARRELL & SCHMIDT, LLP, 445 BROAD HOLLOW ROAD, SUITE 225, MELVILLE, NY, 11747, US

(73) Assignee: Juvent, Inc.

(21) Appl. No.: 11/650,894

(22) Filed: Jan. 8, 2007

(51) Int. Cl. A61B 5/026

(52) U.S. Cl. 600/504

(57) ABSTRACT

The present disclosure provides an apparatus for measuring changes in blood flow. The apparatus includes a band capable of expansion or contraction that is configured for placement around a portion of a subject's body. The apparatus further includes at least one sensing mechanism operatively connected with the band, the at least one sensing mechanism configured to measure and transmit data corresponding to blood flow in the portion of the subject's body.

### BACKGROUND

1. Technical Field

This disclosure relates to the field of medical devices. More particularly, the disclosure relates to a non-invasive apparatus for measuring a patient's blood flow.

2. Description of the Related Art

Blood flow monitors, such as those used to measure blood pressure are well-known. There are two common methods used to measure blood pressure, auscultatory and oscillometric. The ausculatory method typically includes a stethoscope and a sphygmomanometer. A sphygmomanometer or blood pressure meter is a device used to measure blood pressure, comprising an inflatable cuff configured to restrict blood flow, and a manometer to measure the pressure. The oscillometric method utilizes an electronic pressure sensor (transducer) that is fitted into the cuff to detect blood flow, instead of using the stethoscope and the expert's ear. In both cases the cuff is placed on the patient's arm in order to obtain a measurement of the brachial artery. When your pressure is measured, this cuff is tightened to cut off the circulation momentarily. The cuff is loosened, and as the blood begins to flow again, the device measures the systolic and diastolic forces.

However, the blood pressure cuff obstructs the flow of blood, is incapable of sensing very small changes in blood flow and is often uncomfortable. Therefore, what is needed, is a non-invasive blood flow monitor that is configured to sense small changes in blood flow without obstruction.

### SUMMARY

In an embodiment of the present disclosure an apparatus for measuring changes in blood flow is provided. The apparatus includes a band capable of expansion or contraction that is configured for placement around a portion of a subject's body. The apparatus further includes at least one sensing mechanism operatively connected with the band, the at least one sensing mechanism configured to measure and transmit data corresponding to blood flow in the portion of the subject's body.

In one embodiment of the present disclosure a system for measuring changes in blood flow is provided. The system includes a band capable of expansion or contraction configured for placement around a portion of a subject's body. The system also includes at least one sensing mechanism operatively connected with the band, the at least one sensing mechanism configured to measure and transmit data corresponding to blood flow in the portion of the subject's body. The system further includes an analyzer configured to receive data corresponding to the changes in blood flow.

In another embodiment of the present disclosure a method for measuring changes in blood flow is included. The method includes providing a band capable of expansion or contraction that is configured for placement around a portion of a subject's body and subsequently positioning the band on the body. The method also includes utilizing at least one sensing mechanism, which is operatively connected with the band, the at least one sensing mechanism being configured to measure and transmit data and transmitting the measured data to a biological analyzer.

### BRIEF DESCRIPTION OF THE DRAWINGS

Various embodiments of the present disclosure will be described herein below with reference to the figures wherein:

FIG. 1 is a perspective view of an embodiment of the band of the present disclosure;

FIG. 2 is a perspective view of an alternative embodiment of the band of the present disclosure;

FIG. 3 shows a cross-sectional view of the band shown in FIG. 1;

FIG. 4 shows the embodiment shown in FIGS. 1 and 3 placed upon the leg of a user; and

FIG. 5 shows an embodiment of the system of the present disclosure.

### DETAILED DESCRIPTION

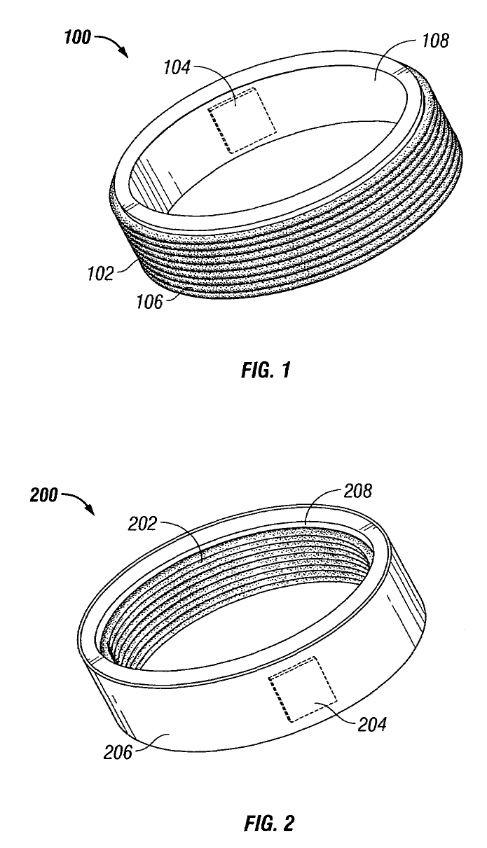

FIG. 1 shows an embodiment of non-invasive blood flow monitor or band 100. Band 100 is configured for placement around the limb of a patient and is capable of measuring changes in blood flow. Band 100 may be constructed out of a number of suitable materials that are capable of expansion and contraction. Some possible materials could include, but are not limited to, semi-rigid plastics, elastomers, textiles and rubbers.

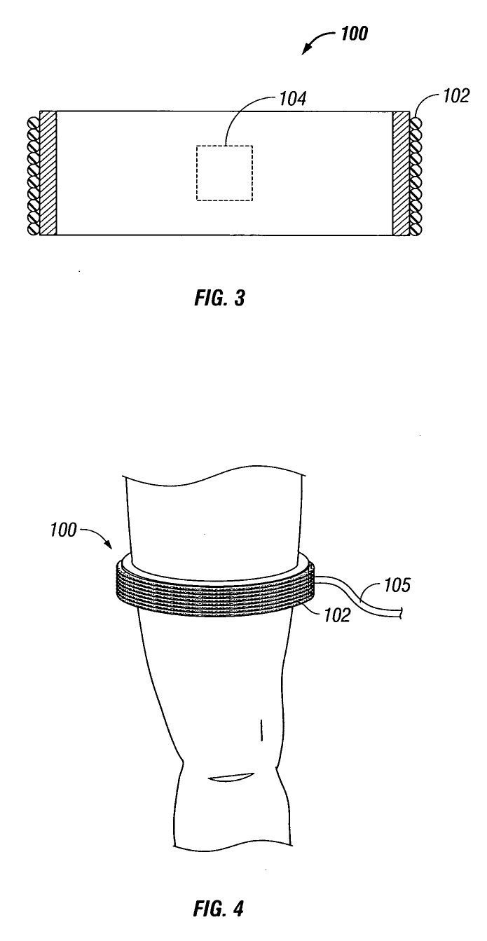

Band 100 may include a layer 102 which may be located on a portion of band 100. Layer 102 may be located either on the exterior of band 100 (FIGS. 1 and 3) or on the interior of band 200 (FIG. 2). It is envisioned that layer 102 may be constructed from a piezoelectric material. A series of piezoelectric fibers could extend in a variety of different configurations on band 100 (e.g. circumferentially). These fibers may be interwoven to form layer 102, or each fiber could extend in a parallel fashion as shown in FIGS. 1 and 2. Moreover, layer 102 may include at least one coating of film located on the exterior portion 106, interior portion 108, or any other portion of band 100. Piezoelectric materials generate a voltage in response to a mechanical stress and will be discussed in greater detail below.

At least one sensing mechanism 104 is operatively connected with band 100. Sensing mechanism 104 is configured to measure and transmit data corresponding to blood flow in a portion of the subject's body. Mechanism 104 may be located adjacent to or within layer 102. Mechanism 104 may include electrical circuitry configured to operatively connect piezoelectric fibers with an analyzer as will be discussed below. Sensing mechanism 104 may interface with piezoelectric materials similar to those available from Advanced Cerametrics, Inc., Lambertville, N.J.

Band 100 collects signals proportional to the change in geometry or deformation of band 100 caused by a proportional change in blood flow. An increase in blood flow would result in expansion of band 100 and a subsequent change in voltage. Sensing mechanism 104 is sensitive to a wide variety of motion, including, but not limited to, muscle movement, blood flow and vibration.

Many pressure sensors display a false signal when they are exposed to vibrations. In order to counteract this, it is envisioned that mechanism 104 may use acceleration compensation elements in addition to the piezoelectric elements discussed above. By carefully matching those elements, the acceleration signal (released from the compensation element) is subtracted from the combined signal of pressure and acceleration to derive the true pressure information.

Piezoelectric fibers may be either bundled or laminated in a parallel array to make transducers or laid out in a flat mono-layer to make actuators. When the fibers are bent, flexed or compressed they generate voltage. The amount of voltage generated is then used to determine blood flow, for example, by accessing a look-up table correlating the amount of voltage generated with blood flow. Alternatively, when the fibers are exposed to an electric field, they mechanically deform; the mechanical deformation can then be used to determine blood flow by visual inspection or by accessing a look-up table correlating amount of mechanical deformation measured for example by millimeters with blood flow.

One possible piezoelectric material, could include polyvinylidene fluoride (PVDF), a piezopolymer, which can be formed in thin films and bonded to different surfaces. The acoustic impedance of piezopolymers is closer to bio tissue and water, and piezopolymers are much less brittle than piezoceramics. Other possible piezoelectric materials, could include, but are not limited to, tourmaline, quartz, topaz, Rochelle salt, quartz analogue crystals, and ceramics with perovskite or tungsten-bronze structures (e.g. BaTiO3, SrTiO3, Pb(ZrTi)O3, KNbO3, LiNbO3, LiTaO3, BiFeO3, NaxWO3, Ba2NaNb5O5 or Pb2KNb5O15). Some specific types of quartz analogue crystals include berlinite (AlPO4) and gallium orthophosphate (GaPO4).

It is contemplated that band 100 may be placed in numerous positions on a patient's body. Band 100 may be placed around a patient's leg, arm, torso, finger, neck, etc. In one embodiment band 100 is placed around a patient's thigh, as shown in FIG. 4. Moreover, multiple bands could be used to determine the blood flow in various parts of the body. The bands could be placed along the path of a vein or artery and used to determine the existence of a blockage or other problem.

As mentioned herein, band 100 may include electrical circuitry configured to transmit data measured at the site to an analyzer, as will be discussed in further detail below. The signals obtained from band 100 may be processed locally and transmitted via telemetry to a receiver in the vicinity. Alternatively, the signals may be hardwired to a processing unit using a cable, or the like, which may be in electrical communication with an analyzer, as will be discussed in further detail below.

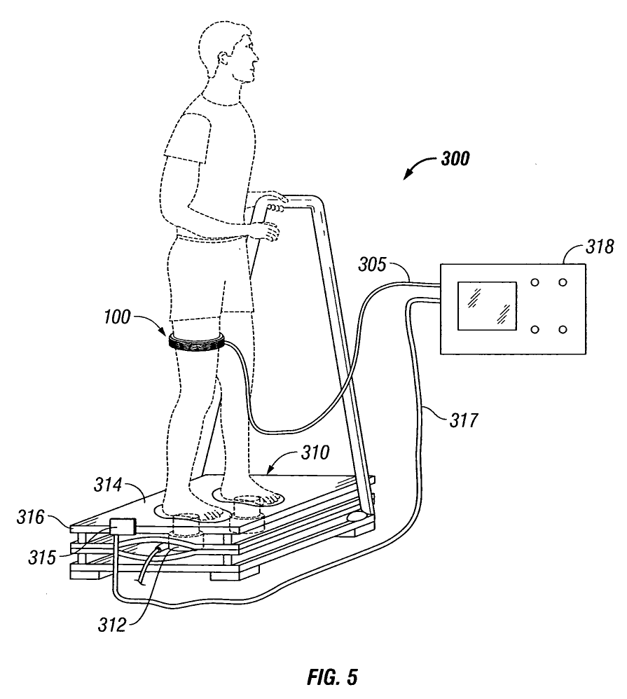

Referring now to FIG. 5, band 100 may be used in accordance with a vibrating system 300. System 300 includes, inter alia, band 100, vibration table 310 and analyzer 318. Vibrations, generated by table 310 for a predetermined period of time, for example, 10 minutes, are transmitted through the patient's body. The vibrations are generated by motorized spring mechanisms 312 located underneath a standing platform 314 of the vibration table 310 and attached thereto. It is contemplated that the vibrations may be generated by a plurality of non-motorized springs or coils attached underneath the standing platform 314, upon which the standing platform 314 rests. It is contemplated that the system of the present disclosure may be carried out while the patient is sitting on the unstable standing platform.

The frequencies imparted by vibration table 310 may be in the range between 30-90 Hz with a peak amplitude between 0.04 and 0.4 g. In certain embodiments, the frequency of the vibration table 310 is approximately 30 Hz and the peak amplitude is 0.2 g. The vibration waves may be sinusoidal, however other waveforms are contemplated. At least one low-mass accelerometer 315 is mounted to vibration table 310 on an outboard side 316 of the standing platform 314. It is contemplated that accelerometer 315 may be mounted to the patient, for example, on the patient's thigh and/or within band 100.

Accelerometer 315 is used to measure the vibrational response of the patient's musculoskeletal system. During the vibration generation of vibration table 310, the response of accelerometer 315 can be amplified by a preamplifier (not shown) as known in the art. It is contemplated that the accelerometer 315 can be worn by the patient.

Thereafter, the vibrational response is measured and recorded by spectrum analyzer/computer 318 which is electrically connected to accelerometer 315 by a cable 317. The accelerometer response data is analyzed to extract information on blood flow. If the accelerometer 315 is attached to the patient, then one can also analyze and extract information on blood flow, blood pressure, circulation or to determine any improvement in the patient's neuro-muscular status.

Analyzer 318 may be a computer, oscilloscope, biomedical monitoring device or any other device having a display. Vibration table 310 may be similar to that shown and described in U.S. Pat. No. 6,607,497, which is incorporated by reference herein. Additional vibration mechanisms are also contemplated. Vibration table 310 could be operated over a wide range of frequencies. In some embodiments the frequency of the vibrations is between approximately 40-60 Hz.

As mentioned hereinabove, analyzer 310 may receive a variety of different signals through band 100. In order to receive data corresponding to a specific signal (e.g. changes in blood flow) analyzer 310 must be configured to differentiate between multiple signals. These signals may correspond to changes in blood flow, muscle movement or vibrations from table 310 or elsewhere. Analyzer 310 may utilize a number of different digital signal processing techniques to extract a particular signal. Some of these techniques may include adaptive filtering techniques, which may use a least mean square (LMS) or recursive mean square (RMS) approach. These techniques enable the extraction of a signal corresponding to a change in blood flow or other desired data. For a detailed discussion on digital signal processing techniques including LMS algorithms for signal extraction from a general signal with periodic interference see C. F. N Cowan and P. M. Grant “Adaptive Filters” Chapter 7, 1985 by Prentice-Hall, Englewood Cliffs, N.J.

In accordance with the present disclosure a method for measuring changes in blood flow is provided. The method includes the step of providing a band capable of expansion or contraction which is configured for placement around a portion of a subject's body (STEP 301) and the step of positioning the band around a particular part of the subject's body (STEP 302). It is envisioned that the band could be placed in a variety of positions on the body including, but not limited to, the legs, arms and torso. The method further includes the step of utilizing at least one sensing mechanism which is operatively connected with the band, the at least one sensing mechanism configured to measure and transmit data (STEP 303). Once the data is measured it is then transmitted to a biological analyzer (STEP 304). The signal corresponding to blood flow may be extracted using the digital signal processing techniques described herein. The method described above may be used in conjunction with a vibration mechanism such as that described in U.S. Pat. No. 6,607,497 and described above.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of preferred embodiments. Those skilled in the art will envision other modifications within the scope and spirit of the claims appended hereto.

1. An apparatus for measuring changes in blood flow comprising:
a band capable of expansion or contraction configured for placement around a portion of a subject's body; and
at least one sensing mechanism operatively connected with the band, the at least one sensing mechanism configured to measure and transmit data corresponding to blood flow in the portion of the subject's body.

2. The apparatus according to claim 1, wherein the at least one sensing mechanism includes a piezoelectric material.

3. The apparatus according to claim 2, wherein the piezoelectric material is selected from the group consisting of polyvinylidene fluoride (PVDF), lead zirconate titanate (PZT), gallium phosphate, tourmaline, quartz, topaz, Rochelle salt, quartz analogue crystals, ceramics with perovskite or tungsten-bronze structures, polymeric materials, yttria stabilized zirconia, silicon carbide, tin oxide, hydroxy apatite, titanium dioxide, aluminum oxide, zirconium diboride and single crystal relaxor materials.

4. The apparatus according to claim 1, wherein the sensing mechanism is configured to sense at least one of muscle movement, blood flow and vibration.

5. The apparatus according to claim 4, wherein the vibration is created by a vibration table in contact with the subject's body.

6. The apparatus according to claim 1, further comprising a communications system configured to transmit data to an analyzer.

7. A system for measuring changes in blood flow comprising:
a band capable of expansion or contraction configured for placement around a portion of a subject's body;
at least one sensing mechanism operatively connected with the band, the at least one sensing mechanism configured to measure and transmit data corresponding to blood flow in the portion of the subject's body; and
an analyzer configured to receive data corresponding to the changes in blood flow.

8. The system according to claim 7, further comprising a vibration mechanism configured to provide vibration to the subject's body.

9. The system according to claim 8, wherein the frequency of the vibration is between approximately 40-60 Hz.

10. The system according to claim 8, wherein the data received by the analyzer includes three distinct signals.

11. The system according to claim 10, wherein the signals correspond to muscle movement, blood flow or vibration.

12. The system according to claim 11, wherein the analyzer utilizes digital signal processing to extract the signals.

13. The system according to claim 12, wherein adaptive filtering techniques are used to extract the signals from the transmitted data.

14. A method for measuring changes in blood flow comprising:
providing a band capable of expansion or contraction which is configured for placement around a portion of a subject's body;
positioning the band around the portion of the subject's body;
utilizing at least one sensing mechanism which is operatively connected with the band, the at least one sensing mechanism configured to measure and transmit data; and
transmitting the measured data to a biological analyzer.

15. The method according to claim 14, further comprising vibrating the subject's body using a vibration mechanism in contact with the subject's body.

16. The method according to claim 14, wherein the at least one sensing mechanism includes a piezoelectric material.

17. The method according to claim 14, wherein the measured data corresponds to at least three signals.

18. The method according to claim 16, wherein the at least three signals reflect muscle motion, blood flow or vibration.

19. The method according to claim 14, further comprising extracting the signal corresponding to blood flow utilizing digital signal processing techniques.
